(19) United States
(12) Patent Application Publication
(10) Pub. No.: US 2008/0219996 A1
(43) Pub. Date: Sep. 11, 2008

(54) Molecular Markers Associated with Bone Metastasis

(76) Inventors: Thea Kalebic, Springfield, NJ (US); Mauro Giulio Papotti, Torino (IT); Giorgio Vittorio Scagliotti, Torino (IT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/066,412

(22) PCT Filed: Sep. 25, 2006
(86) PCT No.: PCT/US06/37193

§ 371 Date: Mar. 11, 2008

Related U.S. Application Data
(60) Provisional application No. 60/720,518, filed on Sep. 26, 2005.

(51) Int. Cl. A61K 39/395; A61K 31/505; A61K 31/506; A61K 31/66

(52) U.S. Cl. 424/142.1; 514/256; 514/75; 514/252.14

(57) ABSTRACT

This invention relates generally to the modulation of expression levels of bone sialoprotein (BSP) in tumors, especially a non-small cell lung cancer tumor, as an indicator of progression to bone metastasis.

### FIELD OF THE INVENTION

This invention relates generally to the modulation of expression levels of bone sialoprotein (BSP) in tumors, especially a non-small cell lung cancer tumor, as an indicator of progression to bone metastasis.

### BACKGROUND OF THE INVENTION

Conventional medical approaches to diagnosis and treatment of disease is based on clinical data alone, or made in conjunction with a diagnostic test. Such traditional practices often lead to therapeutic choices that are not optimal for the efficacy of the prescribed drug therapy or to minimize the likelihood of side effects for an individual subject. Therapy specific diagnostics (a.k.a., theranostics) is an emerging medical technology field, which provides tests useful to diagnose a disease, choose the correct treatment regime and monitor a subject's response. That is, theranostics are useful to predict and assess drug response in individual subjects, i.e., individualized medicine. Theranostic tests are also useful to select subjects for treatments that are particularly likely to benefit from the treatment or to provide an early and objective indication of treatment efficacy in individual subjects, so that the treatment can be altered with a minimum of delay. Theranostics are useful in clinical diagnosis and management of a variety of diseases and disorders, which include, but are not limited to, e.g., cardiovascular disease, cancer, infectious diseases, Alzheimer's Disease and the prediction of drug toxicity or drug resistance. Theranostic tests may be developed in any suitable diagnostic testing format, which include, but is not limited to, e.g., immunohistochemical tests, clinical chemistry, immunoassay, cell-based technologies and nucleic acid tests.

Progress in pharmacogenomics and pharmacogenetics, which establishes correlations between responses to specific drugs and the genetic profile of individual patients and/or their tumors, is foundational to the development of new theranostic approaches. As such, there is a need in the art for the evaluation of patient-to-patient variations.

BSP is an anionic phosphorylated glycoprotein that is expressed almost exclusively in mineralized tissues. BSP has been observed to be expressed by multiple malignant tissues including primary breast cancers, prostate cancer, lung cancer, thyroid cancer, malignant bone disease and neoplastix odontoblasts. There is growing scientific evidence that tumors are heterogeneous and show a different metastatic capacity and affinity for metastasizing to distinct distant organs. For example, lung carcinoma may develop metastasis in the liver, brain or bone. As Paget hypothesized more than a hundred years ago, the affinity for a tumor to metastasize to distant distinct organs is determined by “seed and soil” tumor intrinsic factors and distant organ related factors. BSP has been found to mediate the attachment of tumor cells to collagen type I, which is a major structural component of bone matrix. BSP may increase the invasiveness of tumor cells. Lung cancer is one of the leading causes of cancer related death in the world. The refractoriness of advanced lung cancer to current treatment modalities requires new approaches to reduce mortality and the public health burden associated with this disease. Accordingly, there is a need in the art for additional information about the relationship between modulation of expression levels of BSP and cancer

### SUMMARY OF THE INVENTION

The invention provides a method of predicting the susceptibility of patients having primary lung carcinoma to bone metastasis by identifying increased BSP expression levels.

The invention provides a method for refining diagnosis of bone metastasis in a subject suffering from non-small cell lung carcinoma.

The invention provides a method for guiding a treatment of bone metastasis in a subject suffering from primary lung carcinoma.

The invention further provides a screening method for preventing or treating bone metastasis in a subject suffering from non-small cell lung carcinoma. The increased expression level of BSP is indicative of a propensity of the bone metastasis to develop.

In one embodiment, the subject may be treated with a one or more therapeutic agent which affect bone metastasis and restore bone integrity, such as cathepsin K (CatK) inhibitors, bisphosphonates or RANKL inhibitors and a matrix metalloproteinase inhibitor (MMP) inhibitors.

### BRIEF DESCRIPTION OF THE DRAWINGS

The drawing figures depict preferred embodiments by way of example, not by way of limitations.

FIG. 1: depicts a tissue sample of non-small cell lung carcinoma primary tumor having an increased level of BSP.

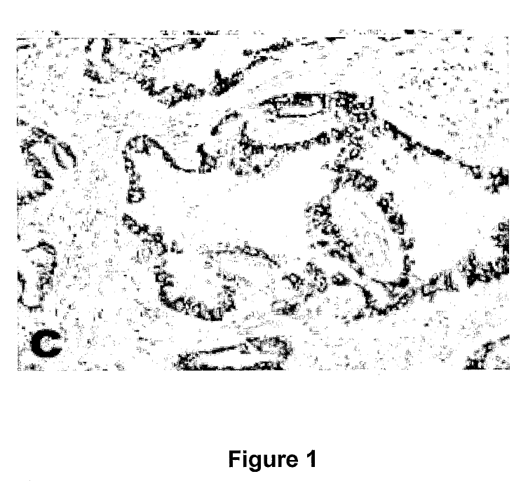

FIG. 2: depicts a tissue sample of non-small cell lung carcinoma primary tumor having no increased level of BSP.

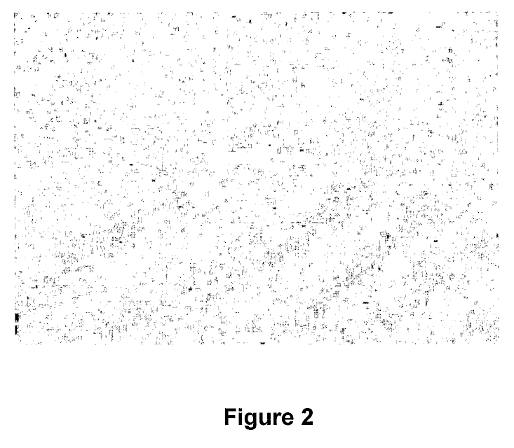

### DETAILED DESCRIPTION OF THE INVENTION

It is to be appreciated that certain aspects, modes, embodiments, variations and features of the invention are described below in various levels of detail in order to provide a substantial understanding of the present invention. In general, the present invention relate to modulation of expression levels of BSP in primary tumor tissue. The various aspects of the present invention further relate to diagnostic/theranostic methods that use the expression levels of BSP of the invention to identify individuals predisposed to bone metastasis or to classify individuals and tumors with regard to drug responsiveness, side effects, or optimal drug dose. Accordingly, various particular embodiments that illustrate these aspects follow.

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description and the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All references cited herein are incorporated herein by reference in their entireties and for all purposes to the same extent as if each individual publication, patent, or patent application was specifically and individually incorporated by reference in its entirety for all purposes.

BSP has been shown to be involved in cell attachment, cell signaling, hydroxyapatite biding, hydroxyapatite nucleation and collagen binding. BSP is highly associated with a distinct type of primary lung tumors which progress to form bone metastasis. Lung cancer, especially non-small cell lung cancer, which progress towards bone metastasis differs at a molecular level from lung cancer which does not develop bone metastasis. BSP has been found increased in all three stages of NSCLC progressing to bone.

Approximately 25% of patients with primary NCSLC develop bone metastasis, but at the time of primary diagnosis of NSCLS it is not possible to predict which individual patients will progress toward bone metastasis. Primary lung carcinoma with propensity to form bone metastasis shows a significant increase in BSP. A statistically significant increase in BSP has been detected in tumors which progress to bone when compared to tumor which do not develop bone metastasis. Increased BSP is shown in 80% of primary lung tumors metastasizing to bone, but only 20% of primary tumors which do not metastasize to bone have a detectable BSP.

Currently, the modalities used to diagnose and characterize primary tumors do not have the ability to predict which tumors will form bone metastasis. The expression of BSP is evaluated by immunohistochemistry, which can be performed manually or by an automated staining system.

Identification of an increase in BSP in primary tumors is a predictor of bone metastasis and as a prognostic factor of shorter survival. BSP assessment can be used in refining the diagnosis, predicting prognosis and guiding therapy of primary tumors.

The invention provides a method of predicting the susceptibility of patients having primary lung carcinoma to bone metastasis by identifying increased BSP expression levels. The method comprise:
(a) detecting an increase level of bone sialoprotein in a subject suffering from primary lung carcinoma that is indicative of a propensity of bone metastasis; and
(b) selecting a treatment to administer a one or more therapeutic agent to the subject with a primary tumor.

The invention provides a method for refining diagnosis of bone metastasis in a subject suffering from non-small cell lung carcinoma. The method for diagnosing a propensity for bone metastasis in a subject having a primary lung carcinoma, comprising the steps of:
(a) detecting an increase level of bone sialoprotein in a subject having primary lung carcinoma that is indicative of a propensity of bone metastasis; and
(b) identifying the subject as having a propensity for bone metastasis.

The invention further provides a screening method identifying the high-risk patients who may benefit from a preventive treatment of bone metastasis in a subject suffering from non-small cell lung carcinoma. Patients can be screened at the time of the initial diagnosis of NSCLC to assess the increased risk of developing bone metastasis, which is indicated by the elevation of BSP. To determine the level of BSP both, a tissue specimen obtained from diagnostic biopsy or a tissue obtained during a surgical therapeutic intervention can be used. The method of choice for evaluating BSP is IHC by using specific antibody against BSP. The increased expression level of BSP in NSCLC is indicative of an increased patients risk for developing the bone metastasis. By using this screening approach, the information about the risk for developing bone metastasis for each individual patient can be obtained in less than 24 hours. High-risk patients will be selected for individualized therapeutical modalities.

In one embodiment, the subject may be treated with a one or more therapeutic agent which affect bone metastasis and restore bone integrity, such as CatK inhibitors, bisphosphonates or RANKL inhibitors or MMP inhibitors.

The one or more therapeutic agent can be administered as a single agent or in combination with the other therapeutic agents of the present invention.

The combination of more than one therapeutic agent of the present invention may be used for simultaneous, sequential or separate use. The combination of certain types of bisphosphonates as described below and certain types of CatK inhibitors as described below may be for simultaneous, sequential or separate use.

The CatK inhibitors used in the pharmaceutical compositions and treatment methods of the present invention typically comprises a compound of formula (V), or a physiologically acceptable and cleavable ester or a salt thereof

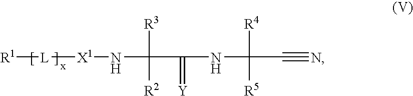

wherein
R¹ is optionally substituted (aryl, aryl-lower alkyl, lower alkenyl, lower alkynyl, heterocyclyl or heterocyclyl-lower alkyl);
R² and R³ together represent lower alkylene, optionally interrupted by O, S or NR⁶, so as to form a ring with the carbon atom to which they are attached, and R⁶ is hydrogen, lower alkyl or aryl-lower alkyl;
R⁴ and R⁵ are independently H, or optionally substituted (lower alkyl or aryl-lower alkyl), —C(O)OR⁷, or —C(O)NR⁷R⁸, wherein R⁷ is optionally substituted (lower alkyl, aryl, aryl-lower alkyl, cycloalkyl, bicycloalkyl, bicycloalkyl or heterocyclyl), and R⁸ is H, or optionally substituted (lower alkyl, aryl, aryl-lower alkyl, cycloalkyl, bicycloalkyl, bicycloalkyl or heterocyclyl), or
R⁴ and R⁵ together represent lower alkylene, optionally interrupted by O, S or NR⁶, so as to form a ring with the carbon atom to which they are attached, and R⁶ is hydrogen, lower alkyl or aryl-lower alkyl; or
R⁴ is H or optionally substituted lower alkyl and R⁵ is a substituent of formula —X²—(Y¹)ₙ—(Ar)ₚ-Q-Z,
   wherein
      Y¹ is O, S, SO, SO₂, N(R⁶)SO₂, N—R⁶, SO₂NR⁶, CONR⁶ or NR⁶CO;
      N is zero or one;
      P is zero or one;
      X² is lower alkylene or when n is zero, X² is also C₂-C₇-alkylene interrupted by O, S, SO, SO₂, NR⁶, SO₂NR⁶, CONR⁶ or NR⁶CO, and R⁶ is hydrogen, lower alkyl or aryl-lower alkyl;
      Ar is arylene;
      Z is hydroxyl, acyloxy, carboxyl, esterified carboxyl, amidated carboxyl, aminosulfonyl, (lower alkyl or aryl-lower alkyl)aminosulfonyl, or (lower alkyl or aryl-lower alkyl)sulfonylaminocarbonyl, or
      Z is tetrazolyl, triazolyl or imidazolyl; and
      Q is a direct bond, lower alkylene, Y¹-lower alkylene or C₂-C₇-alkylene interrupted by Y¹;
X¹ is —C(O)—, —C(S)—, —S(O)—, —S(O)₂— or —P(O)(OR⁶)— and R⁶ is as defined above;
Y is oxygen or sulphur;
L is optionally substituted -Het-, -Het-CH₂— or —CH₂-Het- and Het is a hetero atom selected from O, N or S; and
X is zero or one; and
aryl in the above definitions represents carbocyclic or heterocyclic aryl.

Particular compounds of formula (V) are those wherein R¹ is a substituted phenyl, e.g., whereas the substituent is an optionally substituted nitrogen-containing heterocyclic substituent (=Het^{IV}). This substituent may be at the 2- or 3-position of the phenyl ring, though preferably at the 4-postion. Het^{IV} signifies a heterocyclic ring system containing at least one nitrogen atom, from 2-10, preferably from 3-7, most preferably 4 or 5, carbon atoms and optionally one or more additional heteroatoms selected from O, S or preferably N.

Het^{IV} may comprise an unsaturated, e.g., an aromatic, nitrogen-containing heterocycle; though preferably comprises a saturated nitrogen-containing heterocycle. Particularly preferred saturated nitrogen-containing heterocycles are piperazinyl, preferably piperazin-1-yl, or piperidinyl, preferably piperidin-4-yl.

Het^{IV} may be substituted by one or more substituents, e.g., by up to 5 substituents independently selected from halogen, hydroxy, amino, nitro, optionally substituted C₁-C₄alkyl (e.g., alkyl substituted by hydroxy, alkyloxy, amino, optionally substituted alkylamino, optionally substituted dialkylamino, aryl or heterocyclyl), C₁-C₄alkoxy. Preferably Het^{IV} is substituted at a nitrogen atom, most preferably mono-substituted at a nitrogen atom. Preferred substituents for Het^{IV} are C₁-C₇lower alkyl, C₁-C₇lower alkoxy-C₁-C₇lower alkyl, C₅-C₁₀aryl-C₁-C₇lower alkyl or C₃-C₈cycloalkyl.

Particularly preferred embodiments of the invention provides a compound of formula (VI),

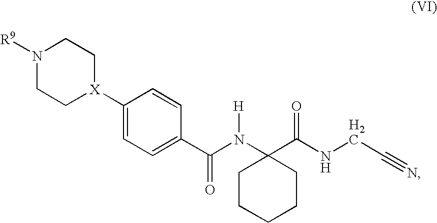

or a pharmaceutically acceptable salt or ester thereof,
wherein
X is CH or N; and
R⁹ is H, C₁-C₇lower alkyl, C₁-C₇lower alkoxy-C₁-C₇lower alkyl, C₅-C₁₀aryl-C₁-C₇lower alkyl or C₃-C₈cycloalkyl.

Thus, particular examples of R⁹ as C₁-C₇lower alkyl are methyl, ethyl, n-propyl or i-propyl are preferred. A particular example of R as C₁-C₇lower alkoxy-C₁-C₇lower alkyl is methoxyethyl. A particular example of R as C₅-C₁₀aryl-C₁-C₇lower alkyl is benzyl. A particular example of R as C₃-C₈cycloalkyl is cyclopentyl. Examples of particular compounds of formula (VI) are:
N-[1-(cyanomethyl-carbamoyl)-cyclohexyl]-4-(piperazin-1-yl)-benzamide;
N-[1-(cyanomethyl-carbamoyl)-cyclohexyl]-4-(4-methyl-piperazin-1-yl)-benzamide;
N-[1-(cyanomethyl-carbamoyl)-cyclohexyl]-4-(4-ethyl-piperazin-1-yl)-benzamide;
N-[1-(cyanomethyl-carbamoyl)-cyclohexyl]-4-[4-(1-propyl)-piperazin-1-yl]-benzamide;
N-[1-(cyanomethyl-carbamoyl)-cyclohexyl]-4-(4-isopropyl-piperazin-1-yl)-benzamide;
N-[1-(cyanomethyl-carbamoyl)-cyclohexyl]-4-(4-benzyl-piperazin-1-yl)-benzamide;
N-[1-(cyanomethyl-carbamoyl)-cyclohexyl]-4-[4-(2-methoxy-ethyl)-piperazin-1-yl]-benzamide;
N-[1-(cyanomethyl-carbamoyl)-cyclohexyl]-4-(1-propyl-piperidin-4-yl)-benzamide;
N-[1-(cyanomethyl-carbamoyl)-cyclohexyl]-4-[1-(2-methoxy-ethyl)-piperidin-4-yl]-benzamide;
N-[1-(cyanomethyl-carbamoyl)-cyclohexyl]-4-(1-isopropyl-piperidin-4-yl)-benzamide;
N-[1-(cyanomethyl-carbamoyl)-cyclohexyl]-4-(1-cyclopentyl-piperidin-4-yl)-benzamide;
N-[1-(cyanomethyl-carbamoyl)-cyclohexyl]-4-(1-methyl-piperidin-4-yl)-benzamide; and
N-[1-(cyanomethyl-carbamoyl)-cyclohexyl]-4-(piperidin-4-yl)-benzamide.

The most preferred CatK inhibitor for use in the invention is N-[1-(cyanomethyl-carbamoyl)-cyclohexyl]-4-[4-(1-propyl)-piperazin-1-yl]-benzamide or a pharmacologically acceptable salt thereof, e.g., hydrogen maleate salt thereof.

All the CatK inhibitors mentioned above are known from the literature.

An alternative class of CatK inhibitors compounds for use in the invention comprises a compound of formula (VII)

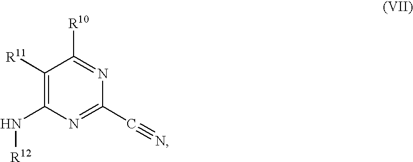

or a physiologically acceptable and cleavable ester or a salt thereof,
wherein
R¹⁰ is H, —R¹⁴, —OR¹⁴ or NR¹³R¹⁴,
wherein
   R¹³ is H, lower alkyl or C₃-C₁₀cycloalkyl; and
   R¹⁴ is lower alkyl or C₃-C₁₀cycloalkyl, and
   R¹³ and R¹⁴ are independently, optionally substituted by halo, hydroxy, lower alkoxy, CN, NO₂ or optionally mono- or di-lower alkyl substituted amino;
R¹¹ is —CO—NR¹⁵R¹⁶, —NH—CO—R¹⁵, —CH₂—NH—C(O)—R¹⁵, —CO—R¹⁵, —S(O)—R¹⁵, —S(O)₂—R¹⁵, —CH₂—CO—R¹⁵ or —CH₂—NR¹⁵R¹⁶,
wherein
   R¹⁵ is aryl, aryl-lower alkyl, C₃-C₁₀cycloalkyl, C₃-C₁₀cycloalkyl-lower alkyl, heterocyclyl or heterocyclyl-lower alkyl;
   R¹⁶ is H, aryl, aryl-lower alkyl, aryl-lower-alkenyl, C₃-C₁₀cycloalkyl, C₃-C₁₀cycloalkyl-lower alkyl, heterocyclyl or heterocyclyl-lower alkyl, or
   R¹⁵ and R¹⁶, together with the nitrogen atom to which they attached, are joined to form an N-heterocyclyl group,
      wherein
         N-heterocyclyl denotes a saturated, partially unsaturated or aromatic nitrogen containing heterocyclic moiety attached via a nitrogen atom thereof having from 3- to 8-ring atoms optionally containing a further 1, 2 or 3 heteroatoms selected from N, NR¹⁷, O, S, S(O) or S(O)₂, wherein R¹⁷ is H or optionally substituted (lower alkyl, carboxy, acyl (including both lower alkyl acyl, e.g., formyl, acetyl or propionyl, or aryl acyl, e.g., benzoyl), amido, aryl, S(O) or S(O)₂), and wherein the N-heterocyclyl is optionally fused in a bicyclic structure, e.g., with a benzene or pyridine ring, and wherein the N-heterocyclyl is optionally linked in a spiro structure with a 3- to 8-membered cycloalkyl or heterocyclic ring wherein the heterocyclic ring has from 3- to 10-ring members and contains from 1-3 heteroatoms selected from N, NR¹⁶, O, S, S(O) or S(O)₂, wherein R¹⁶ is as defined above), and
         heterocyclyl denotes a ring having from 3- to 10-ring members and containing from 1-3 heteroatoms selected from N, NR¹⁷, O, S, S(O) or S(O)₂, wherein R¹⁷ is as defined above); and
   R¹⁵ and R¹⁶ are independently, optionally substituted by one or more groups, e.g., 1-3 groups, selected from halo, hydroxy, oxo, lower alkoxy, CN or NO₂, or optionally substituted (optionally mono- or di-lower alkyl substituted amino, lower-alkoxy, aryl, aryl-lower alkyl, N-heterocyclyl or N-heterocyclyl-lower alkyl (wherein the optional substitution comprises from 1-3 substituents selected from halo, hydroxy, lower alkoxy, lower alkoxy-lower alkyl, lower alkoxy-carbonyl, CN, NO₂, N-heterocyclyl or N-heterocyclyl-lower alkyl, or optionally mono- or di-lower alkyl substituted amino;
R¹² is independently H, or optionally substituted (lower alkyl, aryl, aryl-lower alkyl, C₃-C₁₀cycloalkyl, C₃-C₁₀cycloalkyl-lower alkyl, heterocyclyl or heterocyclyl-lower alkyl); and
R² is optionally substituted by halo, hydroxy, oxo, lower alkoxy, CN, NO₂ or optionally mono- or di-lower alkyl substituted amino; and
   halo or halogen denote I, Br, Cl or F.

The term “lower” referred to above and hereinafter in connection with organic radicals or compounds respectively defines, such as branched or unbranched with up to and including 7, preferably up to and including 5 and advantageously 1, 2 or 3 carbon atoms.

A lower alkyl group is branched or unbranched and contains 1-7 carbon atoms, preferably 1-5 carbon atoms. Lower alkyl represents, e.g., methyl, ethyl, propyl, butyl, isopropyl isobutyl, tertiary butyl or neopentyl (2,2-dimethylpropyl).

Halo-substituted lower alkyl is C₁-C₇-lower alkyl substituted by up to 6 halo atoms.

A lower alkoxy group is branched or unbranched and contains 1-7 carbon atoms, preferably 1-4 carbon atoms. Lower alkoxy represents, e.g., methoxy, ethoxy, propoxy, butoxy, isopropoxy, isobutoxy or tertiary butoxy.

A lower alkene, alkenyl or alkenyloxy group is branched or unbranched and contains 2-7 carbon atoms, preferably 2-4 carbon atoms and contains at least one carbon-carbon double bond. Lower alkene lower alkenyl or lower alkenyloxy represents, e.g., vinyl, prop-1-enyl, allyl, butenyl, isopropenyl or isobutenyl and the oxy equivalents thereof.

A lower alkyne, alkynyl or alkynyloxy group is branched or unbranched and contains 2-7 carbon atoms, preferably 2-4 carbon atoms and contains at least one carbon-carbon triple bond. Lower alkyne or alkynyl represents, e.g., ethynyl, prop-1-ynyl, propargyl, butynyl, isopropynyl or isobutynyl and the oxy equivalents thereof.

In the present description, oxygen containing substituents, e.g., alkoxy, alkenyloxy, alkynyloxy, carbonyl, etc. encompass their sulphur containing homologues, e.g., thioalkoxy, thioalkenyloxy, thioalkynyloxy, thiocarbonyl, sulphone, sulphoxide, etc.

Aryl represents carbocyclic or heterocyclic aryl.

Carbocyclic aryl represents monocyclic, bicyclic or tricyclic aryl, e.g., phenyl or phenyl mono-, di- or tri-substituted by one, two or three radicals selected from lower alkyl, lower alkoxy, aryl, hydroxy, halogen, cyano, trifluoromethyl, lower alkylenedioxy and oxy-C₂-C₃alkylene and other substituents, for instance as described in the examples; or 1- or 2-naphthyl; or 1- or 2-phenanthrenyl. Lower alkylenedioxy is a divalent substituent attached to two adjacent carbon atoms of phenyl, e.g., methylenedioxy or ethylenedioxy. Oxy-C₂-C₃alkylene is also a divalent substituent attached to two adjacent carbon atoms of phenyl, e.g., oxyethylene or oxypropylene. An example for oxy-C₂-C₃alkylene-phenyl is 2,3-dihydrobenzofuran-5-yl.

Preferred as carbocyclic aryl is naphthyl, phenyl or phenyl optionally substituted, for instance, as described in the examples, e.g., mono- or di-substituted by lower alkoxy, phenyl, halogen, lower alkyl or trifluoromethyl.

Heterocyclic aryl represents monocyclic or bicyclic heteroaryl, e.g., pyridyl, indolyl, quinoxalinyl, quinolinyl, isoquinolinyl, benzothienyl, benzofuranyl, benzopyranyl, benzothiopyranyl, furanyl, pyrrolyl, thiazolyl, oxazolyl, isoxazolyl, triazolyl, tetrazolyl, pyrazolyl, imidazolyl, thienyl, or any said radical substituted, especially mono- or di-substituted as defined above.

Preferably, heterocyclic aryl is pyridyl, indolyl, quinolinyl, pyrrolyl, thiazolyl, isoxazolyl, triazolyl, tetrazolyl, pyrazolyl, imidazolyl, thienyl, or any said radical substituted, especially mono- or di-substituted as defined above.

Cycloalkyl represents a saturated cyclic hydrocarbon optionally substituted by lower alkyl which contains 3- to 10-ring carbons and is advantageously cyclopropyl, cyclopentyl, cyclohexyl, cycloheptyl or cyclooctyl optionally substituted by lower alkyl.

N-heterocyclyl is as defined above. Preferred N-heterocyclic substituents are optionally substituted pyrrolidine, pyrrole, diazole, triazole, tetrazole, imidazole, oxazole, thiazole, pyridine, pyrimidine, triazine, piperidine, piperazine, morpholine, phthalimde, hydantoin, oxazolidinone or 2,6-dioxo-piperazine and, e.g., as hereinafter described in the examples.

In a further embodiment the invention provides a compound of formula (VIII),

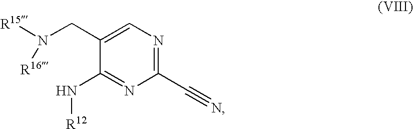

or a pharmaceutically acceptable salt or ester thereof,
wherein

R¹² is as defined above; and

R¹⁵′″ and R¹⁶′″ are as defined above for R¹⁵ and R¹⁶, respectively.

R¹² is preferably R¹²′, which is lower alkyl, e.g., straight chain or more preferably branched-chain C₁-C₆alkyl, e.g., especially 2-ethylbutyl, isobutyl or 2,2-dimethylpropyl or C₃-C₆cycloalkyl, especially cyclopropyl, cyclopentyl or cyclohexyl.

R¹⁵′″ and R¹⁶′″ may be such that R¹⁵′″ and R¹⁶′″, together with the nitrogen atom to which they are joined to, form an N-heterocyclyl group. R¹⁵′″ is preferably optionally substituted (aryl-lower-alkyl, heterocyclyl-aryl, N-heterocyclyl-aryl or aryl-N-heterocyclyl (where N-heterocyclyl is as defined above). R¹⁵′″ is preferably optionally substituted by from 1-4 substituents selected from halo, hydroxy, nitro, cyano, lower-alkyl, lower-alkoxy or lower-alkoxy-lower-alkyl. For example, R¹⁵′″ is 4-methoxy-benzyl, 3-methoxy-benzyl, 4-(4-methyl-piperazin-1-yl)-benzyl, 4-[4-(2-ethoxy-ethyl)-piperazin-1-yl]-benzyl, 1-methyl-1-phenyl-ethyl, 2-(4-methoxy-phenyl)-1,1-dimethyl-ethyl, 2-(4-fluoro-phenyl)-1,1-dimethyl-ethyl, 4-(4-methyl-piperazin-1-yl)-phenyl]-ethyl, 2-[4-(4-isopropyl-piperazin-1-yl)-phenyl]-1,1-dimethyl-ethyl, 2-{4-[4-(2-methoxy-ethyl)-piperazin-1-yl]-phenyl}-1,1-dimethyl-ethyl, 2-{3-[4-(2-ethoxy-ethyl)-piperazin-1-yl]-phenyl}-1,1-dimethyl-ethyl, 2-[3-(4-ethyl-piperazin-1-yl)-phenyl]-1,1-dimethyl-ethyl, 2-[3-(4-isopropyl-piperazin-1-yl)-phenyl]-1,1-dimethyl-ethyl, 1,1-dimethyl-2-(3-pyrrolidin-1-yl-phenyl)-ethyl, 2-{3-[4-(2-methoxy-ethyl)-piperazin-1-yl]-phenyl}-1,1-dimethyl-ethyl, 2-(4-methoxy-phenyl)-ethyl, 2-[4-(4-methyl-piperazin-1-yl)-phenyl]-ethyl, 2-[4-(4-isopropyl-piperazin-1-yl)-phenyl]-ethyl, 2-{4-[4-(2-methoxy-ethyl)-piperazin-1-yl]-phenyl}-ethyl, 2-(3-methoxy-phenyl)-ethyl, 2-[3-(4-methyl-piperazin-1-yl)-phenyl]-ethyl, 2-[4-(4-isopropyl-piperazin-1-yl)-phenyl]-ethyl, 2-pyrrol-1-yl-ethyl, 3-piperidin-1-yl-propyl, 2-(4-methoxy-phenyl)-2-methyl-propyl, 2-methyl-2-[4-(4-methyl-piperazin-1-yl)-phenyl]-propyl, 2-[4-(4-isopropyl-piperazin-1-yl)-phenyl]-2-methyl-propyl, 2-{4-[4-(2-ethoxy-ethyl)-piperazin-1-yl]-phenyl}-2-methyl-propyl, 2-{4-[pyrimidin-1-yl]-phenyl}-2-methyl-propyl, 4-(3-methoxy-phenyl)-piperazin-1-yl-methyl, 4-(4-methoxy-phenyl)-piperazin-1-yl-methyl, 1-methyl-1-(1-phenyl-cyclopropyl)-ethyl. For example, R¹⁵′″ and R¹⁶′″, together with the nitrogen atom to which they are joined to, form an N-heterocyclyl group is 4-(2-pyridin-4-yl-ethyl)-piperazin-1-yl, [4-(2-pyridin-2-yl-ethyl)-piperazin-1-yl, 4-pyridin-4-ylmethyl-piperazin-1-yl, 4-(2-piperidin-1-yl-ethyl)-piperazin-1-yl, 4-(2-pyrrolidin-1-yl-ethyl)-piperazin-1-yl, 4-(2-Diethylamino-ethyl)-piperazin-1-yl, 4-(3-diethylamino-propyl)-piperazin-1-yl, 4-(1-methyl-piperidin-4-yl)-piperazin-1-yl, 4-pyrrolidin-1-yl-piperidin-1-yl, 4-(2-methoxy-ethyl)-piperazin-1-yl.

In a preferred embodiment, the invention provides a compound of formula (IX)

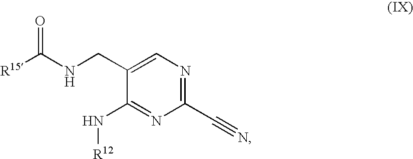

or a pharmaceutically acceptable salt or ester thereof,
wherein

R¹² is as defined above; and

R¹⁵′ is as defined above for R¹⁵.

R¹² is preferably R¹²′ which is lower alkyl, e.g., straight chain or more preferably branched-chain C₁-C₆alkyl, e.g., especially 2-ethylbutyl, isobutyl or 2,2-dimethylpropyl or C₃-C₆cycloalkyl, especially cyclopropyl, cyclopentyl or cyclohexyl.

R¹⁵′ is preferably optionally substituted (aryl-lower-alkyl, heterocyclyl-aryl, N-heterocyclyl-aryl or aryl-N-heterocyclyl (where N-heterocyclyl is as defined above). R¹⁵′ is preferably optionally substituted by from 1-4 substituents selected from halo, hydroxy, nitro, cyano, lower-alkyl, lower-alkoxy, lower-alkoxy-carbonyl or lower-alkoxy-lower-alkyl. For example, R¹⁵′ is 4-methoxy-phenyl, 4-(1-propyl-piperidin-4-yl)-phenyl, 4-(4-methyl-piperazin-1-yl)-phenyl, 4-[1-(2-methoxy-ethyl)-piperidin-4-yl]-phenyl, 4-(4-propyl-piperazin-1-yl)-phenyl, 3-[4-(4-methyl-piperazin-1-yl)-phenyl]-propionyl, 3-[3-(4-methyl-piperazin-1-yl)-phenyl]-propionyl, 4-(4-ethyl-piperazin-1-yl)-phenyl, 4-(4-isopropyl-piperazin-1-yl)-phenyl, 4-[4-(2-ethoxy-ethyl)-piperazin-1-yl]-phenyl, 4-[4-(2-methoxy-ethyl)-piperazin-1-yl]-phenyl, 4-piperazin-1-yl-phenyl, 4-[4-(carboxylic acid tert-butyl ester) piperazino-1-yl-]-phenyl, 3-[4-(carboxylic acid tert-butyl ester) piperazino-1-yl-]-phenyl, 3-(4-methyl-piperazin-1-yl)-phenyl, 3-(4-ethyl-piperazin-1-yl)-phenyl, 3-(4-isopropyl-piperazin-1-yl)-phenyl, 3-[4-(2-methoxy-ethyl)-piperazin-1-yl]-phenyl, 3-[4-(2-ethoxy-ethyl)-piperazin-1-yl]-phenyl, 3-(2-pyrrolidin-1-yl-ethoxy)-phenyl, 3-(2-dimethylamino-ethoxy)-4-methoxy-phenyl, 4-dimethylaminomethyl-phenyl, 4-(4-methyl-piperazin-1-ylmethyl)-phenyl, 4-[1-(2-methoxy-ethyl)-piperidin-4-ylmethyl]-phenyl, 4-methoxy-3-(2-piperidin-1-yl-ethoxy)-phenyl, 3-[4-(4-ethyl-piperazin-1-yl)-phenyl]-2,2-dimethyl-propionyl, 3-[4-(4-propyl-piperazin-1-yl)-phenyl]-propionyl, 3-(4-pyrrolidin-1-yl-phenyl)-propionyl, 3-[3-(4-ethyl-piperazin-1-yl)-phenyl]-2,2-dimethyl-propionyl, 3-{3-[4-(2-methoxy-ethyl)-piperazin-1-yl]-phenyl}-2,2-dimethyl-propionyl, 3-{3-[4-(2-ethoxy-ethyl)-piperazin-1-yl]-phenyl}-2,2-dimethyl-propionyl, 3-(3-pyrrolidin-1-yl-phenyl)-propionyl, 2-[4-(4-methyl-piperazin-1-yl)-phenyl]-isobutyl, 2-(4-methoxy-phenyl)-acetyl, 2-(3-methoxy-phenyl)-acetyl, 2-[4-(4-methyl-piperazin-1-yl)-phenyl]-acetyl, 2-[4-(4-ethyl-piperazin-1-yl)-phenyl]-acetyl, 2-[4-(4-isopropyl-piperazin-1-yl)-phenyl]-acetyl, 2-(4-pyrrolidin-1-yl-phenyl)-acetyl, 2-[4-(2-diethylamino-ethylamino)-phenyl]-isobutyl, 2-(4-pyrrolidin-1-yl-phenyl)-isobutyl.

Particularly preferred compounds are
N-[2-cyano-4-(2,2-dimethyl-propylamino)-pyrimidin-5-ylmethyl]-4-(1-propyl-piperidin-4-yl)-benzamide;
N-[2-cyano-4-(2,2-dimethyl-propylamino)-pyrimidin-5-ylmethyl]-4-(4-methyl-piperazin-1-yl)-benzamide,
N-[2-cyano-4-(2,2-dimethyl-propylamino)-pyrimidin-5-ylmethyl]-4-[1-(2-methoxy-ethyl)-piperidin-4-yl]-benzamide;
N-[2-cyano-4-(2,2-dimethyl-propylamino)-pyrimidin-5-ylmethyl]-4-(4-propyl-piperazin-1-yl)-benzamide;
N-[2-cyano-4-(2,2-dimethyl-propylamino)-pyrimidin-5-ylmethyl]-2,2-dimethyl-3-[4-(4-methyl-piperazin-1-yl)-phenyl]-propionamide;
N-[2-cyano-4-(2,2-dimethyl-propylamino)-pyrimidin-5-ylmethyl]-2,2-dimethyl-3-[3-(4-methyl-piperazin-1-yl)-phenyl]-propionamide;
N-[2-cyano-4-(2,2-dimethyl-propylamino)-pyrimidin-5-ylmethyl]-4-(4-ethyl-piperazin-1-yl)-benzamide;
N-[2-cyano-4-(2,2-dimethyl-propylamino)-pyrimidin-5-ylmethyl]-4-(4-isopropyl-piperazin-1-yl)-benzamide;
N-[2-cyano-4-(2,2-dimethyl-propylamino)-pyrimidin-5-ylmethyl]-4-[4-(2-ethoxy-ethyl)-piperazin-1-yl]-benzamide;
N-[2-cyano-4-(2,2-dimethyl-propylamino)-pyrimidin-5-ylmethyl]-4-[4-(2-methoxy-ethyl)-piperazin-1-yl]-benzamide;
N-[2-cyano-4-(2,2-dimethyl-propylamino)-pyrimidin-5-ylmethyl]-4-piperazin-1-yl-benzamide;
4-(4-{[2-cyano-4-(2,2-dimethyl-propylamino)-pyrimidin-5-ylmethyl]-carbamoyl}-phenyl)-piperazine-1-carboxylic acid tert-butyl ester;
4-(3-{[2-cyano-4-(2,2-dimethyl-propylamino)-pyrimidin-5-ylmethyl]-carbamoyl}-phenyl)-piperazine-1-carboxylic acid tert-butyl ester;
N-[2-cyano-4-(2,2-dimethyl-propylamino)-pyrimidin-5-ylmethyl]-3-(4-methyl-piperazin-1-yl)-benzamide;
N-[2-cyano-4-(2,2-dimethyl-propylamino)-pyrimidin-5-ylmethyl]-3-(4-ethyl-piperazin-1-yl)-benzamide;
N-[2-cyano-4-(2,2-dimethyl-propylamino)-pyrimidin-5-ylmethyl]-3-(4-isopropyl-piperazin-1-yl)-benzamide;
N-[2-cyano-4-(2,2-dimethyl-propylamino)-pyrimidin-5-ylmethyl]-3-[4-(2-methoxy-ethyl)-piperazin-1-yl]-benzamide;
N-[2-cyano-4-(2,2-dimethyl-propylamino)-pyrimidin-5-ylmethyl]-3-[4-(2-ethoxy-ethyl)-piperazin-1-yl]-benzamide;
N-[2-cyano-4-(2,2-dimethyl-propylamino)-pyrimidin-5-ylmethyl]-4-methoxy-3-(2-pyrrolidin-1-yl-ethoxy)-benzamide;
N-[2-cyano-4-(2,2-dimethyl-propylamino)-pyrimidin-5-ylmethyl]-3-(2-dimethylamino-ethoxy)-4-methoxy-benzamide;
N-[2-cyano-4-(2,2-dimethyl-propylamino)-pyrimidin-5-ylmethyl]-4-dimethylaminomethyl-benzamide;
N-[2-cyano-4-(2,2-dimethyl-propylamino)-pyrimidin-5-ylmethyl]-4-(4-methyl-piperazin-1-ylmethyl)-benzamide;
N-[2-cyano-4-(2,2-dimethyl-propylamino)-pyrimidin-5-ylmethyl]-4-[1-(2-methoxy-ethyl)-piperidin-4-ylmethyl]-benzamide;
N-[2-cyano-4-(2,2-dimethyl-propylamino)-pyrimidin-5-ylmethyl]-4-methoxy-3-(2-piperidin-1-yl-ethoxy)-benzamide; and
N-[2-cyano-4-(2,2-dimethyl-propylamino)-pyrimidin-5-ylmethyl]-3-[4-(4-ethyl-piperazin-1-yl)-phenyl]-2,2-dimethyl-propionamide,
   or pharmaceutically acceptable salts thereof.

Especially preferred pharmaceutically acceptable salts of the CatK inhibitors are maleate salts, e.g., N-[1-(cyanomethyl-carbamoyl)-cyclohexyl]-4-(1-propyl-piperidin-4-yl)-benzamide hydrogen maleate.

In one embodiment, the CatK inhibitor is N-[2-cyano-4-(2,2-dimethyl-propylamino)-pyrimidin-5-ylmethyl]-4-(4-methyl-piperazin-1-yl)-benzamide.

The bisphosphonates for use in the present invention are preferably N-bisphosphonates.

For the purposes of the present description an N-bisphosphonate is a compound which in addition to the characteristic geminal bisphosphate moiety comprises a nitrogen containing side chain, e.g., a compound of formula (I)

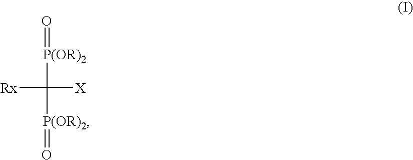

wherein
X is hydrogen, hydroxyl, amino, alkanoyl, or an amino group substituted by C₁-C₄alkyl or alkanoyl;
R is hydrogen or C₁-C₄alkyl; and
Rx is a side chain which contains an optionally substituted amino group, or a nitrogen containing heterocycle (including aromatic nitrogen-containing heterocycles,
   and pharmaceutically acceptable salts thereof or any hydrate thereof.

Thus, e.g., suitable N-bisphosphonates for use in the invention may include the following compounds or a pharmaceutically acceptable salt thereof, or any hydrate thereof: 3-amino-1-hydroxypropane-1,1-diphosphonic acid (pamidronic acid), e.g., pamidronate (APD); 3-(N,N-dimethylamino)-1-hydroxypropane-1,1-diphosphonic acid, e.g., dimethyl-APD; 4-amino-1-hydroxybutane-1,1-diphosphonic acid (alendronic acid), e.g., alendronate; 1-hydroxy-3-(methylpentylamino)-propylidene-bisphosphonic acid, ibandronic acid, e.g., ibandronate; 6-amino-1-hydroxyhexane-1,1-diphosphonic acid, e.g., amino-hexyl-BP; 3-(N-methyl-N-n-pentylamino)-1-hydroxypropane-1,1-diphosphonic acid, e.g., methyl-pentyl-APD (=BM 21.0955); 1-hydroxy-2-(imidazol-1-yl)ethane-1,1-diphosphonic acid, e.g., zoledronic acid; 1-hydroxy-2-(3-pyridyl)ethane-1,1-diphosphonic acid (risedronic acid), e.g., risedronate, including N-methylpyridinium salts thereof, e.g., N-methylpyridinium iodides, such as NE-10244 or NE-10446; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g., EB 1053 (Leo); 1-(N-phenylaminothiocarbonyl)methane-1,1-diphosphonic acid, e.g., FR 78844 (Fujisawa); 5-benzoyl-3,4-dihydro-2H-pyrazole-3,3-diphosphonic acid tetraethyl ester, e.g., U-81581 (Upjohn); and 1-hydroxy-2-(imidazo[1,2-a]pyridin-3-yl)ethane-1,1-diphosphonic acid, e.g., YM 529.

In one embodiment a particularly preferred N-bisphosphonate for use in the invention comprises a compound of formula (II)

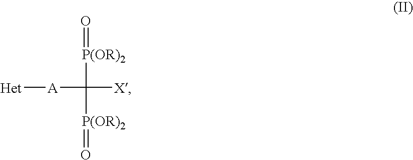

wherein
Het is an imidazole, oxazole, isoxazole, oxadiazole, thiazole, thiadiazole, pyridine, 1,2,3-triazole, 1,2,4-triazole or benzimidazole radical, which is optionally substituted by alkyl, alkoxy, halogen, hydroxyl, carboxyl, an amino group optionally substituted by alkyl or alkanoyl radicals or a benzyl radical optionally substituted by alkyl, nitro, amino or aminoalkyl;
A is a straight-chained or branched, saturated or unsaturated hydrocarbon moiety containing from 1-8 carbon atoms;
X′ is a hydrogen atom, optionally substituted by alkanoyl, or an amino group optionally substituted by alkyl or alkanoyl radicals; and
R is a hydrogen atom or an alkyl radical,
   and the pharmacologically acceptable salts thereof.

In a further embodiment a particularly preferred bisphosphonate for use in the invention comprises a compound of formula (III)

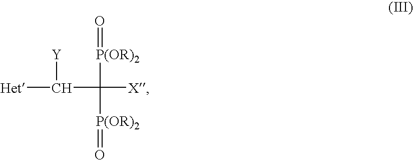

wherein
Het′ is a substituted or unsubstituted heteroaromatic five-membered ring selected from the group consisting of imidazolyl, imidazolinyl, isoxazolyl, oxazolyl, oxazolinyl, thiazolyl, thiazolinyl, triazolyl, oxadiazolyl and thiadiazolyl wherein said ring can be partly hydrogenated and wherein said substituents are selected from at least one of the group consisting of C₁-C₄alkyl, C₁-C₄alkoxy, phenyl, cyclohexyl, cyclohexylmethyl, halogen and amino and wherein two adjacent alkyl substituents of Het can together form a second ring;
Y is hydrogen or C₁-C₄alkyl;
X″ is hydrogen, hydroxyl, amino or an amino group substituted by C₁-C₄alkyl; and
R is hydrogen or C₁-C₄alkyl,
   as well as the pharmacologically acceptable salts and isomers thereof.

In a yet further embodiment a particularly preferred bisphosphonate for use in the invention comprises a compound of formula (IV)

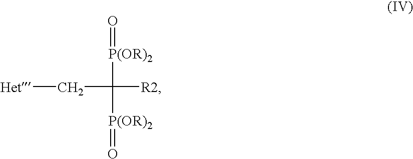

wherein
Het′″ is an imidazolyl, 2H-1,2,3-, 1H-1,2,4- or 4H-1,2,4-triazolyl, tetrazolyl, oxazolyl, isoxazolyl, oxadiazolyl, thiazolyl or thiadiazolyl radical which is unsubstituted or C-mono- or di-substituted by lower alkyl, by lower alkoxy, by phenyl which may in turn be mono- or di-substituted by lower alkyl, lower alkoxy and/or halogen, by hydroxy, by di-lower alkylamino, by lower alkylthio and/or by halogen and is N-substituted at a substitutable N-atom by lower alkyl or by phenyl-lower alkyl which may in turn be mono- or di-substituted in the phenyl moiety by lower alkyl, lower alkoxy and/or halogen; and
R2 is hydrogen, hydroxy, amino, lower alkylthio or halogen,
   lower radicals having up to and including 7 C-atoms, or a pharmacologically acceptable salt thereof.

Examples of particularly preferred N-bisphosphonates for use in the invention are:
2-(1-methylimidazol-2-yl)-1-hydroxyethane-1,1-diphosphonic acid;
2-(1-benzylimidazol-2-yl)-1-hydroxyethane-1,1-diphosphonic acid;
2-(1-methylimidazol-4-yl)-1-hydroxyethane-1,1-diphosphonic acid;
1-amino-2-(1-methylimidazol-4-yl)ethane-1,1-diphosphonic acid;
1-amino-2-(1-benzylimidazol-4-yl)ethane-1,1-diphosphonic acid;
2-(1-methylimidazol-2-yl)ethane-1,1-diphosphonic acid;
2-(1-benzylimidazol-2-yl)ethane-1,1-diphosphonic acid;
2-(imidazol-1-yl)-1-hydroxyethane-1,1-diphosphonic acid;
2-(imidazol-1-yl)ethane-1,1-diphosphonic acid;
2-(4H-1,2,4-triazol-4-yl)-1-hydroxyethane-1,1-diphosphonic acid;
2-(thiazol-2-yl)ethane-1,1-diphosphonic acid;
2-(imidazol-2-yl)ethane-1,1-diphosphonic acid;
2-(2-methylimidazol-4(5)-yl)ethane-1,1-diphosphonic acid;
2-(2-phenylimidazol-4(5)-yl)ethane-1,1-diphosphonic acid;
2-(4,5-dimethylimidazol-1-yl)-1-hydroxyethane-1,1-diphosphonic acid; and
2-(2-methylimidazol-4(5)-yl)-1-hydroxyethane-1,1-diphosphonic acid,
   and pharmacologically acceptable salts thereof.

The most preferred N-bisphosphonate for use in the invention is 2-(imidazol-1yl)-1-hydroxyethane-1,1-diphosphonic acid (zoledronic acid) or a pharmacologically acceptable salt thereof, in particular, zoledronic acid.

The N-bisphosphonates may be used in the form of an isomer or of a mixture of isomers where appropriate, typically as optical isomers, such as enantiomers or diastereoisomers or geometric isomers, typically cis-trans isomers. The optical isomers are obtained in the form of the pure antipodes and/or as racemates.

The N-bisphosphonates can also be used in the form of their hydrates or include other solvents used for their crystallization.

Especially preferred pharmaceutically acceptable salts of the N-bisphosphonates are those where one, two, three or four, in particular, one or two, of the acidic hydrogens of the bisphosphonic acid are replaced by a pharmaceutically acceptable cation, in particular, sodium, potassium or ammonium, in first instance sodium.

The RANKL inhibitors of the present invention include agents that target, decrease or inhibit RANK/RANKL pathway. RANK inhibitors prevent osteoclast-mediated bone loss in a range of conditions including osteoporosis, treatment-induced bone loss (bone loss due to glucocorticoid treatment and immunosuppression), rheumatoid arthritis, bone metastases and multiple myeloma. An example of a RANKL inhibitor includes, but is not limited to, denosumab.

The MMP inhibitors, of the present invention include a compound which targets, decreases or inhibits a class of protease enzyme that selectively catalyze the hydrolysis of polypeptide bonds including the enzymes MMP-2 and MMP-9 that are involved in promoting the loss of tissue structure around tumors and facilitating tumor growth, angiogenesis and metastasis. A target of a MMP inhibitor includes, but is not limited to, polypeptide deformylase. Example of a MMP inhibitor include, but are not limited to, actinonin, which is also known as butanediamide, N4-hydroxy-N1-[(1S)-1-[[(2S)-2-(hydroxymethyl)-1-pyrrolidinyl]carbonyl]-2-methylpropyl]-2-pentyl-, (2R)-(9Cl); epigallocatechin gallate; collagen peptidomimetic and non-peptidomimetic inhibitors; tetracycline derivatives, e.g., hydroxamate peptidomimetic inhibitor batimastat; and its orally-bioavailable analogue marimastat, prinomastat, metastat, neovastat, tanomastat, TAA211, MMI270B or AAJ996.

The following EXAMPLES are presented in order to more fully illustrate the preferred embodiments of the invention. These EXAMPLES should in no way be construed as limiting the scope of the invention, as defined by the appended claims.

### EXAMPLE 1

Increased BSP expression has a predictive value and distinguishes bone metastasis progressors from non-bone metastasis progressors

A total of 60 primary NSCLC specimens derived from surgically resected tissue are analyzed for the expression of BSP. The total is divided into two groups. One group includes 30 cases consisting of primary NSCLC tumors with a propensity to develop bone metastasis. The second group includes 30 controls, consisting of primary NSCLC which do not form bone metastasis. The two groups are matched for clinical stage, type of tumor, sex and age of the patients.

### I. Time to Progression to Bone Metastasis

Among the total of 30 patients which progressed toward bone metastasis, 70% of patients formed bone metastasis within 12 months. Twenty-one (21) out of the 30 patients formed bone metastasis within 12 months. Ninety percent (90%) of patients developed bone metastasis within 24 months. Twenty-seven (27) out of 30 patients developed bone metastasis within 24 months. Three patients have progressed to bone metastasis after 24 months. In the control group, none of 30 patients developed bone metastasis. To detect bone metastasis bone scans were performed.

II. BSP in Bone Metastasis Progressors Vs. Non-Progressors

Levels of BSP are assessed by IHC as follows:

Tumor tissue sections are deparaffinized in fresh xylen substitute (isoparaffin ISODIAF—Baker) and hydrated in absolute ethanol for 10 minutes, followed by the additional soaking in 96% ethanol for 10 minutes. The tissue sections are then rinsed in 70% ethanol. The tissue sections are then washed 2× for 5 minutes in phosphate buffered saline, having a pH of about 7.4, w/o Ca, Mg (PBS). Antigen retrieval is done by using MW citrate buffer (pH about 6) for 3 cycles 5′ each, then equilibrating the section at RT in PBS 1×.

Subsequently, the endogenous peroxidase is blocked with fresh solution of 3.0% H₂O₂ in methanol for 30 minutes, quickly rinsing tissue sections in PBS and washing twice in PBS, 5 minutes each time.

Unspecific absorption is reduced by incubating the tissue sections with BSA (bovine serum albumin) 1% and Tween20 0.04% in PBS for 30 minutes.

After such a preparation, tissue sections are incubated with primary anti-BSP 1:1000 diluted in antibody diluent (DakoCytomation) (approximately 200 μL) in humid chamber at 4° C. overnight. In order to visualize the binding of the primary antibody, sections are incubated with Envision™ (DakoCytomation) for 40 minutes at room temperature. Tissue sections are then washed twice for 5 minutes in PB and incubated with freshly prepared DAB solution (DakoCytomation) for 10 minutes at room temperature. Finally tissue sections are rinsed thoroughly in water.

Hematoxylin is used for 30 seconds. The sections are dehydrated (ethanol→ISODIAF) and then coversliped with ULTRAKIT (Baker) and dried at room temperature.

Among the total of 30 patients which progressed toward bone metastasis, 80% (24 out of 30 patients which have progressed toward bone metastasis) in course of their disease, show increased BSP levels at the time of the diagnosis of the primary NSCLC. Among the control group, 7 out of 30 patients have shown some degree of BSP staining.

### III. NSCLC Stage I, II and III Progressors Show Increased BSP

Seven patients having Stage I tumors, 5 patients having Stage II tumors and 18 patients having Stage III tumors were evaluated. A control group of patients which did not have bone metastasis progressing tumors was also evaluated. The control group of patients consisted of a comparable number of respective tumor stage: 7 patients having tumors Stage I, 5 patients having tumors Stage II and 18 patients having tumors Stage III. To determine the stage, as defined by growth and spread of non-small cell lung cancer (NSCLC) the TNM staging system was used, also known as the American Joint Committee on Cancer (AJCC) system.

Histologic subtypes of NSCLC progressors have increased BSP.

BSP has been found increased in multiple histologic subtypes of NSCLC with propensity to develop bone metastasis (23 adenocarcinoma, 6 squamose carcinoma and 1 tumor with adeno-squamose features). Non-progressive control group of NSCLC consisted of 19 adenocarcinoma, 7 squamose carcinoma, 3 bronchioloalveolar carcinoma, I adenosquamose tumor).

### EXAMPLE 2

BSP is associated with worse prognosis and shorter survival.

Clinical follow-up of patients after the diagnosis and surgical removal of the tumor provided the following information about the progression of the disease, metastases formation and survival. A total of 27 BSP-positive patients and 27 BSP-negative patients have been observed for a duration of 1 year. Among BSP-positive patients one year survival rate was 70%, while survival rate for BSP-negative patients was 89%. Three years survival rate for BSP-positive patients was 35%. In contrast, for BSP-negative patients three years survival rate was 76%.

### EQUIVALENTS

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled.

1: A method for treating bone metastasis in a subject suffering from primary lung carcinoma, comprising the steps of:
(a) detecting an increase level of bone sialoprotein in a subject suffering from primary lung carcinoma that is indicative of a propensity of bone metastasis; and
(b) administering a one or more therapeutic agent to the subject, wherein the one or more therapeutic agent is selected from a CatK inhibitor, a bisphosphonate, a RANKL inhibitor and a MMP inhibitor.

2: The method of claim 1, wherein the CatK inhibitor is of the formula (VII) 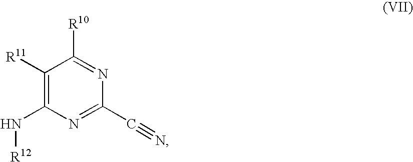
wherein
R¹⁰ is H, —R¹⁴—OR¹⁴ or NR¹³R¹⁴;
wherein
R¹³ is H, lower alkyl or C₃-C₁₀cycloalkyl;
R¹⁴ is lower alkyl or C₃-C₁₀cycloalkyl;
R¹³ and R¹⁴ are independently, optionally substituted by halo, hydroxy, lower alkoxy, CN, NO₂ or optionally mono- or di-lower alkyl substituted amino;
R¹¹ is —CO—NR¹⁵R¹⁶, —NH—CO—R¹⁵, —CH₂—NH—C(O)—R¹⁵, —CO—R¹⁵, —S(O)—R¹⁵, —S(O)₂—R¹⁵, —CH₂—CO—R¹⁵ or —CH₂—NR¹⁵R¹⁶;
wherein
R¹⁵ is aryl, aryl-lower alkyl, C₃-C₁₀cycloalkyl, C₃-C₁₀-cycloalkyl-lower alkyl, heterocyclyl or heterocyclyl-lower alkyl;
R¹⁶ is H, aryl, aryl-lower alkyl, aryl-lower-alkenyl, C₃-C₁₀cycloalkyl, C₃-C₁₀cycloalkyl-lower alkyl, heterocyclyl or heterocyclyl-lower alkyl;
R¹⁵ and R¹⁶, together with the nitrogen atom to which they attached are joined to, form an N-heterocyclyl group,
wherein
N-heterocyclyl denotes a saturated, partially unsaturated or aromatic nitrogen containing heterocyclic moiety attached via a nitrogen atom thereof having from 3- to 8-ring atoms optionally containing a further 1, 2 or 3 heteroatoms selected from N, NR¹⁷, O, S, S(O) or S(O)₂, wherein R¹⁷ is H or optionally substituted lower alkyl, carboxy, acyl (including both lower alkyl acyl, or aryl acyl; amido, aryl, S(O) or S(O)₂);
N-heterocyclyl is optionally fused in a bicyclic structure selected from a benzene or pyridine ring;
N-heterocyclyl is optionally linked in a spiro structure with a 3- to 8-membered cycloalkyl or heterocyclic ring wherein the heterocyclic ring has from 3- to 10-ring members and contains from 1-3 heteroatoms selected from N, NR¹⁶, O, S, S(O) or S(O)₂, wherein R¹⁶ is as defined above);
heterocyclyl denotes a ring having from 3- to 10-ring members and containing from 1-3 heteroatoms selected from N, NR¹⁷, O, S, S(O) or S(O)₂, wherein R¹⁷ is as defined above;
R¹⁵ and R¹⁶ are independently, optionally substituted by one or more groups selected from halo, hydroxy, oxo, lower alkoxy, CN or NO₂, or optionally substituted mono- or di-lower alkyl substituted amino, lower-alkoxy, aryl, aryl-lower alkyl, N-heterocyclyl or N-heterocyclyl-lower alkyl wherein the optional substitution comprises from 1-3 substituents selected from halo, hydroxy, lower alkoxy, lower alkoxy-lower alkyl, lower alkoxy-carbonyl, CN, NO₂, N-heterocyclyl or N-heterocyclyl-lower alkyl, or optionally mono- or di-lower alkyl substituted amino;
R¹² is independently H, or optionally substituted lower alkyl, aryl, aryl-lower alkyl, C₃-C₁₀cycloalkyl, C₃-C₁₀cycloalkyl-lower alkyl, heterocyclyl or heterocyclyl-lower alkyl; and
R² is optionally substituted by halo, hydroxy, oxo, lower alkoxy, CN, NO₂ or optionally mono- or di-lower alkyl substituted amino; and
halo or halogen denote 1, Br, Cl or F; or a pharmaceutically acceptable salt.

3: The method of claim 1, wherein the bisphosphonate is a compound of formula (IV) 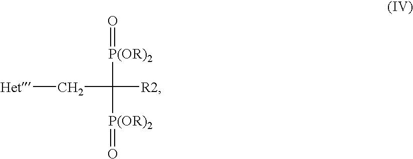
wherein
Het′″ is an imidazolyl, 2H-1,2,3-, 1H-1,2,4- or 4H-1,2,4-triazolyl, tetrazolyl, oxazolyl, isoxazolyl, oxadiazolyl, thiazolyl or thiadiazolyl radical which is unsubstituted or C-mono- or di-substituted by lower alkyl, by lower alkoxy, by phenyl which may in turn be mono- or di-substituted by lower alkyl, lower alkoxy and/or halogen, by hydroxy, by di-lower alkylamino, by lower alkylthio and/or by halogen and is N-substituted at a substitutable N-atom by lower alkyl or by phenyl-lower alkyl which may in turn be mono- or di-substituted in the phenyl moiety by lower alkyl, lower alkoxy and/or halogen;
R² is hydrogen, hydroxy, amino, lower alkylthio or halogen, lower radicals having up to and including 7 C-atoms;
and pharmaceutically acceptable salts thereof.

4: The method of claim 1, wherein the RANKL inhibitor is denosumab.

5: The method of claim 1, wherein the MMP inhibitor is selected from marimastat, prinomastat, metastat, neovastat or tanomastat.

6: The method of claim 1, wherein the CatK inhibitor is N-[2-cyano-4-(2,2-dimethyl-propylamino)-pyrimidin-5-ylmethyl]-4-(4-methyl-piperazin-1-yl)-benzamide.

7: The method of claim 1, wherein the bisphosphonate is 2-(imidazol-1yl)-1-hydroxyethane-1,1-diphosphonic acid.
